(19) United States
(12) Patent Application Publication
(10) Pub. No.: US 2021/0393593 A1
(43) Pub. Date: Dec. 23, 2021

(54) USE OF HISTIDINE, GLYCINE AND OTHER AMINO ACIDS FOR PREVENTING INSULIN RESISTANCE AND/OR DIABETES

(71) Applicant: SOCIETE DES PRODUITS NESTLE S.A., Vevey (CH)

(72) Inventors: Francois-Pierre Martin, Vuisternens-devant-Romont (CH); Jorg Hager, Houtaud (FR); Jonathan Pinkney, Plymouth Devon (GB); Joanne Hosking, Plymouth Devon (GB)

(21) Appl. No.: 17/279,694

(22) PCT Filed: Sep. 24, 2019
(86) PCT No.: PCT/EP2019/075640

§ 371 Date: Mar. 25, 2021

(30) Foreign Application Priority Data
Sep. 27, 2018 (EP) 18197379.3

(51) Int. Cl. A61K 31/4172; A61K 31/198; A61P 3/10

(52) U.S. Cl. CPC A61K 31/4172; A61P 3/10; A61K 31/198

(57) ABSTRACT

The invention relates to compositions and methods using a combination of at least one histidine or derivative thereof, at least one glycine or derivative thereof, and at least one additional agent selected from N-acetyl-cysteine, lysine, or arginine. An aspect of the invention is a method of promoting healthy fat metabolism and metabolic health in children at risk of developing insulin resistance and diabetes, promoting healthy lipolysis and use of fatty acid in metabolism, promoting healthy fat and adipocyte metabolism during puberty and adolescence, treating or preventing oxidative stress, a condition associated with oxidative stress, a reduced level of glutathione, or a condition associated with a reduced level of glutathione, by administering an effective amount of a combination of at least one glycine or derivative thereof, and lysine.

### INTRODUCTION

With 1.8 billion adolescents worldwide (25% world pop.), and about 42 million children under 5 overweight or obese in 2013, management of pre-diabetes and Type-2 Diabetes (T2D) in childhood and adolescence has become critical. Nutrition has a pivotal role to play since both pre-diabetes and T2D are largely preventable and closely linked to lifestyle, dietary intake and exercise.

In addition, (Pre-)Diabetes in children differs from adults in many physiological and metabolic aspects, including insulin, sexual maturity & growth, neurologic vulnerability to hypoglycemia, and ability to provide self-care. However, compared to adult studies, there is less data in children, in whom insulin resistance (IR) is subject to marked variations, being particularly influenced by pubertal timing as well as both changing body composition and physical activity. In youths, the significance of pubertal IR is open to debate whilst the understanding of the underlying mechanisms that link obesity and IR is incomplete. Whereas IR relates to the resistance to insulin-mediated glucose uptake in insulin-sensitive tissues, childhood and pubertal IR may well result from various metabolic and physiological requirements, including the effects of increased growth hormone secretion (either direct and/or via the action of IGF-1) (Pinkney, Streeter et al. 2014).

In the context of metabolic health, childhood and adolescence, obesity introduces a significant disturbance into normal growth and pubertal patterns (Sandhu et al., 2006; Marcovecchio and Chiarelli, 2013). Recent analysis from the Earlybird study has demonstrated the important influences on IR of age and gender in puberty (Jeffery S et al. Pediatric Diabetes, 2017), which differs in many ways with the adult phenotype (Jeffery et al., 2012). The study exemplified how IR starts to rise in mid-childhood, some years before puberty, with more than 60% of the variation in IR prior to puberty remaining unexplained. In addition, conventional markers to detect diabetes, and to identify individuals at high risk of developing diabetes, and for adult metabolic disease risk, such as HbA1c, lose sensitivity and specificity for pediatric applications, suggesting that other factors influence the variance of these markers in youths (Hosking et al., 2014).

One potentially important factor currently being studied is the role of excess body weight during childhood. This can also influence pubertal development through effects on timing of pubertal onset and hormone levels (Marcovecchio and Chiarelli, 2013). The interactions of adiposity with puberty is complex and gender-specific. Moreover, in girls, higher level of IR limit further gain in body fat in the long term—an observation potentially consistent with the concept of IR as a mechanism of insulin desensitization as an adaptive response to weight gain (Hosking et al., 2011). Recently, weight gain and impaired glucose metabolism were shown to be predicted by inefficient subcutaneous fat cell lipolysis (Arner, Andersson et al. 2018). Adipocyte mobilization of fatty acids (lipolysis) is instrumental for energy expenditure. Lipolysis displays both spontaneous (basal) and hormone-stimulated activity. Thus, inefficient lipolysis (high basal/low stimulated) is linked to future weight gain and impaired glucose metabolism and may constitute a treatment target.

The role of resting energy expenditure and weight gain in children is subject to controversy, with particular interest in studying the influence of puberty on long term body composition. Obesity develops when energy intake is greater than energy expenditure, the excess energy being stored mainly as fat in adipose tissue. Body weight loss and prevention of weight gain can be achieved by reducing energy intake or bioavailability, increasing energy expenditure, and/or reducing storage as fat. However, overweight subjects or subjects at risk of becoming overweight often need nutritional assistance for better managing their body weight, e.g. through increasing satiety and/or reducing body weight gain.

To address these particular evidence gaps, the EarlyBird study was designed as a longitudinal cohort study of healthy children with the express intent to investigate the influences of anthropometric, clinical and metabolic processes on glucose and insulin metabolism during childhood and adolescence. The EarlyBird cohort is a non-interventional prospective study of 300 healthy UK children followed-up annually throughout childhood. The investigators tackled the challenging task of integrating and correlating the temporal variations of these different data types in the Earlybird childhood cohort from age 5 to age 20, including anthropometric, clinical and serum biomarker (metabonomic) data.

The present inventors observed that only few and specific amino acid and lipid-derived metabolites were associated with IR development throughout childhood and adolescence in this cohort of healthy children. The population of children overweight or obese at age 5, further developed excessive fat mass gain and body weight gain throughout puberty and adolescence, and have higher HOMA-IR than in other children. In the Earlybird cohort, overweight children at age 5 remain overweight throughout childhood, and will acquire a high IR status from age 10 during pubertal development and development of additional fat mass. The present inventors identified negative association with creatine, glycine, histidine, lysine, and arginine status, which may be indicative of potential deregulation of oxidative stress and adipocyte lipolysis during growth and development, concomitant or contributing to IR development.

Without being bound by theory, the present inventors noted that glutathione disulfide (GSSG) is reduced to glutathione (GSH) which functions in cellular reduction-oxidation (redox) reactions, and thus GSH has the potential to prevent damage mediated by reactive oxygen species (ROS) in a physiological way compared to traditional antioxidant supplementation. However, maintaining reduced GSH in cells under conditions of ROS stress is critical to provide health benefits. The present inventors believe that a combination of histidine and glycine, with either lysine, or arginine or N-acetylcysteine (NAC) can provide a GSH-salvage mechanism and a superior redox potential compared to merely increasing total glutathione. Further, in this regard, the combination of these compounds may have additional effects to promote healthy fat and lean mass metabolism during growth and development.

### Definitions

Various terms used throughout the specification are defined as shown below.

The following terms are used throughout the specification to describe the different early life stages of a subject of the invention, particularly a human subject:
Infant, Newborn: a human subject during the first month after birth;
Infant: a human subject between 1 and 23 months of age inclusive;
Child, Preschool: a human subject between the ages of 2 and 5 inclusive, i.e. from the subject's 2^{nd} birthday up to and including the day before their 6^{th} birthday;
Child: a human subject between the ages of 6 and 12 inclusive;
Prepuberty: age 6 or 7 of a human subject;
Mid-childhood: age 7 or 8 of a human subject; and
Adolescent (or adolescence): a human subject between the ages of 13 and 18 inclusive (the corresponding early life stage in other subjects, for example in dogs, would be between the ages 6 months to 18 months inclusive)
Adulthood: 19 years old and above

The various metabolites mentioned throughout the specification are also known by other names.

The metabolite “Histidine” is also known as S)-4-(2-amino-2-Carboxyethyl)imidazole; (S)-alpha-amino-1H-Imidazole-4-propanoic acid; (S)-alpha-amino-1H-Imidazole-4-propionic acid; (S)-1H-Imidazole-4-alanine; (S)-2-amino-3-(4-Imidazolyl)propionsaeure; (S)-Histidine; (S)1H-Imidazole-4-alanine; 3-(1H-Imidazol-4-yl)-L-alanine; amino-1H-Imidazole-4-propanoate; amino-1H-Imidazole-4-propanoic acid; amino-4-lmidazoleproprionate; amino-4-Imidazoleproprionic acid; Glyoxaline-5-alanine.

The metabolite “Glycine” is also known as Aminoacetic acid; Aminoessigsaeure; Aminoethanoic acid; Glycocoll; Glykokoll; Glyzin; Leimzucker; 2-Aminoacetate; amino-Acetic acid; Glicoamin; Glycolixir; Glycosthene; Gyn-hydralin; Padil.

The metabolite “Lysine” is also known as (S)-2,6-Diaminohexanoic acid; (S)-alpha,epsilon-Diaminocaproic acid; (S)-Lysine; 6-ammonio-L-Norleucine; L-2,6-Diaminocaproic acid; L-Lysin; Lysina; Lysine acid; Lysinum; (S)-2,6-Diaminohexanoate; (+)-S-Lysine; (S)-2,6-diamino-Hexanoate; (S)-2,6-diamino-Hexanoic acid; (S)-a,e-Diaminocaproate; (S)-a,e-Diaminocaproic acid; 2,6-Diaminohexanoate; 2,6-Diaminohexanoic acid; 6-amino-Aminutrin; 6-amino-L-Norleucine; a-Lysine; alpha-Lysine; Aminutrin; L-2,6-Diainohexanoate; L-2,6-Diainohexanoic acid; Enisyl MeSH.

The metabolite “Arginine” is also known as (2S)-2-amino-5-(carbamimidamido)Pentanoic acid; (2S)-2-amino-5-Guanidinopentanoic acid; (S)-2-amino-5-Guanidinopentanoic acid; (S)-2-amino-5-Guanidinovaleric acid; L-(+)-Arginine; (S)-2-amino-5-[(Aminoiminomethyl)amino]-pentanoate; (S)-2-amino-5-[(Aminoiminomethyl)amino]-pentanoic acid; (S)-2-amino-5-[(Aminoiminomethyl)amino]pentanoate; (S)-2-amino-5-[(Aminoiminomethyl)amino]pentanoic acid; 2-amino-5-Guanidinovalerate; 2-amino-5-Guanidinovaleric acid; 5-[(Aminoiminomethyl)amino]-L-norvaline; L-a-amino-D-Guanidinovalerate; L-a-amino-D-Guanidinovaleric acid; L-alpha-amino-delta-Guanidinovalerate; L-alpha-amino-delta-Guanidinovaleric acid; N5-(Aminoiminomethyl)-L-ornithine.

The term Insulin resistance (IR) is a pathological condition in which cells fail to respond normally to the hormone insulin. The body produces insulin when glucose starts to be released into the bloodstream from the digestion of carbohydrates (primarily) in the diet. Under normal conditions of insulin reactivity, this insulin response triggers glucose being taken into body cells, to be used for energy, and inhibits the body from using fat for energy, thereby causing the concentration of glucose in the blood to decrease as a result, staying within the normal range even when a large amount of carbohydrates is consumed. During insulin resistance, however, excess glucose is not sufficiently absorbed by cells even in the presence of insulin, thereby causing an increase in the level of blood sugar. IR is one of the factors involved in type 2 Diabetes and Pre-diabetes.

IR can be diagnosed through different means:
Fasting insulin levels: A fasting serum insulin level greater than 25 mIU/L or 174 pmol/L is considered insulin resistance
Glucose tolerance test and Matsuda index
Homeostatic Model Assessment (HOMA), the normal reference range for HOMA-IR differs depending on ethnicity and gender, and must be defined for each population.
Quantitative insulin sensitivity check index (QUICKI)
Hyperinsulinemic euglycemic clamp
Modified insulin suppression test

The term “pre-diabetes” describes a condition in which fasting blood glucose levels are equal or higher than 5.6 mmol/L of blood plasma, although not high enough to be diagnosed with type 2 diabetes. Pre-diabetes has no signs or symptoms. People with pre-diabetes have a higher risk of developing type 2 diabetes and cardiovascular (heart and circulation) disease. Without sustained lifestyle changes, including healthy eating, increased activity and losing weight, approximately one in three people with pre-diabetes will go on to develop type 2 diabetes. There are two pre-diabetic conditions:
Impaired glucose tolerance (IGT) is where blood glucose levels are equal or higher than 5.6 mmol/L of blood plasma but not high enough to be classified as diabetes. Impaired glucose tolerance is defined as two-hour glucose levels of 140 to 199 mg per dL (7.8 to 11.0 mmol) on the 75-g oral glucose tolerance test, so levels for diabetes is above 11 mmol in ogtt.
Impaired fasting glucose (IFG) is where blood glucose levels are escalated in the fasting state but not high enough to be classified as diabetes. Impaired fasting glucose is defined as glucose levels of 100 to 125 mg per dL (5.6 to 6.9 mmol per L) in fasting patients. So diabetes is above 6.9 mmol.
It is possible to have both Impaired Fasting Glucose (IFG) and Impaired Glucose Tolerance (IGT).

As used herein, the term “reference value” can be defined as the average value measured in biofluid samples of a substantially healthy normal glycaemic population. Said population may have an average fasting glucose level of less than 5.6 mmol/L. The average age of said population is preferably substantially the same as that of the subject. The average BMI sds of said population is preferably substantially the same as that of the subject. The average physical activity level of said population is preferably substantially the same as that of the subject. Said population may be of substantially the same race as the human subject. Said population may number at least 2, 5, 10, 100, 200, 500, or 1000 individuals. Said population may be substantially the same breed when the subject is a pet.

The term “high levels of glucose” or “high glucose levels” is defined as equal to or higher than 5.6 mmol/L as measured in a biofluid sample of a subject.

The term “biofluid” can be, for example, human blood (particularly human blood serum, human blood plasma), urine or interstitial fluids.

“Overweight” is defined for an adult human as having a BMI between 25 and 30. “Body mass index” or “BMI” means the ratio of weight in kg divided by the height in metres, squared. “Obesity” is a condition in which the natural energy reserve, stored in the fatty tissue of animals, in particular humans and other mammals, is increased to a point where it is associated with certain health conditions or increased mortality. “Obese” is defined for an adult human as having a BMI greater than 30. “Normal weight” for an adult human is defined as a BMI of 18.5 to 25, whereas “underweight” may be defined as a BMI of less than 18.5. Body mass index (BMI) is a measure used to determine childhood overweight and obesity in children and teens. Overweight in children and teens is defined as a BMI at or above the 85th percentile and below the 95th percentile for children and teens of the same age and sex. Obesity is defined as a BMI at or above the 95th percentile for children and teens of the same age and sex. Normal weight in children and teens is defined as a BMI at or above the 5th percentile and below the 85th percentile for children and teens of the same age and sex. Underweight in children and teens is defined as below the 5th percentile for children and teens of the same age and sex. BMI is calculated by dividing a person's weight in kilograms by the square of height in meters. For children and teens, BMI is age- and sex-specific and is often referred to as BMI-for-age. A child's weight status is determined using an age- and sex-specific percentile for BMI rather than the BMI categories used for adults. This is because children's body composition varies as they age and varies between boys and girls. Therefore, BMI levels among children and teens need to be expressed relative to other children of the same age and sex.

The term “subject” is preferably a human subject or can be a pet subject e.g. a cat a dog. In one embodiment, the subject is a male subject. In one embodiment, the subject is a female subject.

The term “substantially” is taken to mean 50% or greater, more preferably 75% or greater, or more preferably 90% or greater. The term “about” or “approximately” when referring to a value or to an amount or percentage is meant to encompass variations of in some embodiments ±20%, in some embodiments ±10%, in some embodiments ±5%, in some embodiments ±1%, in some embodiments ±0.5%, and in some embodiments ±0.1% from the specified value, amount or percentage.

### DETAILED DESCRIPTION

The present invention also relates to a composition for use in promoting metabolic health in a subject at risk of developing insulin resistance.

The present invention also relates to a composition for use in promoting metabolic health in a subject at risk of developing diabetes.

The present invention also relates to a composition for use in promoting metabolic health in a subject at risk of developing insulin resistance and diabetes.

The present invention also relates to a composition for use in preventing insulin resistance in a subject.

The present invention also relates to a composition for use in preventing an increase in insulin resistance in a subject.

The present invention also relates to a composition for use in preventing or treating diabetes in a subject.

The present invention also relates to a composition for use in (i) preventing or preventing an increase in insulin resistance; and (ii) preventing or treating diabetes, in a subject.

In one embodiment, the subject is a human subject. In one embodiment, the human subject is a child. In one embodiment, the human subject is an adolescent. In one embodiment, the human subject is an adult.

The composition comprises at least one histidine or derivative thereof.

The composition comprises at least one histidine or derivative thereof, at least one glycine or derivative thereof, and optionally at least one additional agent, selected from N-acetyl-cysteine, lysine, or arginine, or derivative of said additional agents.

In some embodiments, the composition comprises at least one histidine, at least one glycine, and at least one additional agent, selected from N-acetyl-cysteine, lysine, or arginine.

In some embodiments, the composition comprises at least one histidine, at least one glycine, and at least two additional agents, selected from N-acetyl-cysteine, lysine, or arginine.

In one embodiment, the composition comprises at least one histidine, at least one glycine, and additional agents N-acetyl-cysteine, lysine, and arginine.

In some embodiments, the composition is for use in treating or preventing at least one additional physical state as described herein.

In some embodiments, the composition is for use in treating or preventing at least one additional physical state, wherein said physical state is an inflammatory disease. In one embodiment, said inflammatory disease is treated or prevented in an adolescent male. In one embodiment, said adolescent male is aged 13 or 14 years. In one embodiment, said composition comprises at least one histidine or derivative thereof, at least one glycine or derivative, and optionally at least one lysine.

In some embodiments, the composition comprises a combination of at least one histidine or derivative thereof, at least one glycine or derivative thereof, and at least one lysine or derivative thereof for use in treating or preventing at least one physical state selected from the group consisting of inefficient lipolysis, such as high basal lipolysis, low stimulated lipolysis, or a condition associated with inefficient lipolysis.

In some embodiments, the composition comprises a combination of at least one histidine or derivative thereof, at least one glycine or derivative thereof, and lysine for use in promoting and maintaining efficient subcutaneous fat cell lipolysis and fatty acid metabolism.

In some embodiments, the composition comprises a combination of at least one histidine or derivative thereof, at least one glycine or derivative thereof, and lysine for use in treating or preventing at least one physical state selected from the group consisting of high HOMA-IR, high fasting glucose and high insulin.

In some embodiments, the composition comprises a combination of at least one histidine or derivative thereof, at least one glycine or derivative thereof, and lysine for use in treating or preventing at least one physical state selected from the group consisting of oxidative stress, a condition associated with oxidative stress, or a condition associated with a reduced level of glutathione.

In some embodiments, the composition comprises a combination of at least one histidine or derivative thereof, at least one glycine or derivative thereof, and lysine for use in treating or preventing at least one physical state selected from the group consisting of high body weight gain and associated disturbed glucose metabolism during growth and development, high body fat gain and associated disturbed glucose metabolism during growth and development, high central adiposity and associated disturbed glucose metabolism during growth and development.

In another embodiment, the composition comprises a combination of at least one histidine or derivative thereof, at least one glycine or derivative thereof and at least one lysine or derivative thereof for use in enhancing metabolization of reactive oxygen species, improving glucose control and/or improving muscle function in an individual with at least one of obesity or diabetes.

In another embodiment, the composition comprises a combination of at least one histidine or derivative thereof, at least one glycine or derivative thereof and at least one arginine or derivative thereof for use in enhancing metabolization of reactive oxygen species, improving glucose control and/or improving muscle function in an individual with at least one of obesity or diabetes.

In another embodiment, the composition comprises a combination of at least one histidine or derivative thereof, at least one glycine or derivative thereof and at least one N-acetyl-cysteine or derivative thereof for use in enhancing metabolization of reactive oxygen species, improving glucose control and/or improving muscle function in an individual with at least one of obesity or diabetes.

In another embodiment, the composition comprises a combination of at least one histidine or derivative thereof, at least one glycine or derivative thereof and at least one lysine or derivative thereof for use in improving mitochondrial function in an individual with sarcopenia. The individual with sarcopenia can be otherwise healthy or obese sarcopenic.

In another embodiment, the composition comprises a combination of at least one histidine or derivative thereof, at least one glycine or derivative thereof and at least one arginine or derivative thereof for use in improving mitochondrial function in an individual with sarcopenia. The individual with sarcopenia can be otherwise healthy or obese sarcopenic.

In another embodiment, the composition comprises a combination of at least one histidine or derivative thereof, at least one glycine or derivative thereof and at least one N-acetyl-cysteine or derivative thereof for use in improving mitochondrial function in an individual with sarcopenia. The individual with sarcopenia can be otherwise healthy or obese sarcopenic.

In an embodiment, the composition of the invention is for use in treating or preventing at least one physical state selected from the group consisting of deleterious effects of type I diabetes, type II diabetes, complications from diabetes, insulin resistance, metabolic syndrome, dyslipidemia, overweight, obesity, raised cholesterol levels, raised triglyceride levels, elevated fatty acid levels, fatty liver disease, cardiovascular disease, myopathy such as statin-induced myopathy, non-alcoholic steatohepatitis, hypertension, atherosclerosis/coronary artery disease, myocardial damage after stress.

In an embodiment, the composition is a combination of at least one histidine or derivative thereof, at least one glycine or derivative thereof, and at least one additional agent, selected from N-acetyl-cysteine, lysine, or arginine for use according to the invention via oral administration.

In an embodiment, the composition is a combination of at least one histidine or derivative thereof, at least one glycine or derivative thereof, and at least one additional agent, selected from N-acetyl-cysteine, lysine, or arginine is administered for use according to the invention in a food product.

In an embodiment, the composition is a combination of at least one histidine or derivative thereof, at least one glycine or derivative thereof, and N-acetyl-cysteine or functional derivative thereof wherein a dipeptide provides at least a portion of the at least one glycine or functional derivative thereof and the at least one N-acetylcysteine or functional derivative thereof for use according to the invention.

Each of the compounds can be administered at the same time as the other compounds (i.e., as a single unit) or separated by a time interval (i.e., in separate units).

In an embodiment, the composition is a combination of at least one histidine or derivative thereof, at least one glycine or derivative thereof, and at least one lysine or derivative thereof for use according to the invention via administration in the same composition as a single unit.

In an embodiment, the composition is a combination of at least one histidine or derivative thereof, at least one glycine or derivative thereof, and at least one arginine or derivative thereof for use according to the invention via administration in the same composition as a single unit.

In an embodiment, the composition is combination of at least one histidine or derivative thereof, at least one glycine or derivative thereof, and at least one N-acetylcysteine or derivative thereof for use according to the invention via administration in the same composition as a single unit.

In an embodiment, the composition is a combination of at least one histidine or derivative thereof, at least one glycine or derivative thereof, and at least one lysine or derivative thereof for use according to the invention via administration in separate units.

In an embodiment, the composition is a combination of at least one histidine or derivative thereof, at least one glycine or derivative thereof, and at least one arginine or derivative thereof for use according to the invention via administration in separate units.

In an embodiment, the composition is a combination of at least one histidine or derivative thereof, at least one glycine or derivative thereof, and at least one N-acetylcysteine or derivative thereof for use according to the invention via administration in separate units.

The composition of the invention comprises the combination in an amount effective for use in at least one of (i) subcutaneous fat cell lipolysis and use of fatty acid in metabolism, inefficient lipolysis (high basal/low stimulated), a condition associated with inefficient lipolysis, (ii) high HOMA-IR, high fasting glucose and insulin, (iii) oxidative stress, a condition associated with oxidative stress, or a condition associated with a reduced level of glutathione, (iv) high body weight gain and associated disturbed glucose metabolism during growth and development, high body fat gain and associated disturbed glucose metabolism during growth and development, high central adiposity and associated disturbed glucose metabolism during growth and development.

In some embodiments, the composition of the invention comprises the combination in an amount effective for use in at least one of (i) a condition associated with inefficient lipolysis, (ii) a condition associated with high IR, (iii) a condition associated with oxidative stress, (iv) a condition associated with high body weight gain and associated disturbed glucose metabolism during growth and development.

In an embodiment, the composition of the invention is a food product for use according to the invention.

The invention further relates to a kit comprising at least one histidine or derivative thereof, at least one glycine or derivative thereof, and at least one of the following amino acids, lysine, arginine or N-acetyl-cysteine, or derivative thereof of said amino acids for admixing to form one or more of the compositions disclosed herein and/or for use according to the invention, for example in separate containers as two or more liquid solutions or dried powders. In some embodiments, one or more of these compounds can be isolated compounds.

The combination of at least one glycine or derivative thereof and at least one N-acetylcysteine or functional derivative thereof can be provided by any of the compositions disclosed by U.S. Pat. Nos. 8,362,080, 8,802,730 and 9,084,760, each entitled “Increasing glutathione levels for therapy,” and WO2016/191468 entitled “Benefits of Supplementation with N-Acetylcysteine and Glycine to Improve Glutathione Levels,” each incorporated herein by reference in its entirety.

Accordingly, an aspect of the present invention is a composition comprising at least one histidine or derivative thereof, at least one glycine or derivative thereof, and at least one of the following amino acid, lysine, arginine or N-acetyl-cysteine, or derivative thereof in an amount effective for use in the treatment or prevention of at least condition selected from the group consisting of deleterious effects of (i) subcutaneous fat cell lipolysis and use of fatty acid in metabolism, inefficient lipolysis (high basal/low stimulated), a condition associated inefficient lipolysis, or (ii) high HOMA-IR, high fasting glucose and insulin, or (iii) oxidative stress, a condition associated with oxidative stress, or a condition associated with a reduced level of glutathione, or (iv) high body weight gain and associated disturbed glucose metabolism during growth and development, high body fat gain and associated disturbed glucose metabolism during growth and development, high central adiposity and associated disturbed glucose metabolism during growth and development.

The present invention also relates in general to a method for promoting metabolic health, particularly in a subject at risk of developing insulin resistance and/or diabetes.

The present invention also relates to a method for preventing or preventing an increase in insulin resistance.

The present invention also relates to a method for preventing or treating diabetes.

The present invention also relates to a method for (i) preventing or preventing an increase in insulin resistance; and (ii) preventing or treating diabetes.

In one embodiment, the subject is a human subject. In one embodiment, the human subject is a child. In one embodiment, the human subject is an adolescent. In one embodiment, the human subject is an adult.

The present invention also relates in general to a method for promoting healthy fat metabolism, in a human subject at risk of developing insulin resistance and diabetes.

The method of the invention comprises administration of a combination of at least one histidine or derivative thereof, at least one glycine or derivative thereof, and optionally at least one additional agent, selected from N-acetyl-cysteine, lysine, or arginine.

In some embodiments, the method is for treating or preventing inefficient lipolysis, such as high basal lipolysis, low stimulated lipolysis, or a condition associated with inefficient lipolysis, said method comprising administering an effective amount of a combination of at least one histidine or derivative thereof, at least one glycine or derivative thereof, and lysine.

In some embodiments, the method for promoting healthy fat metabolism includes promoting and maintaining healthy subcutaneous fat cell lipolysis and fatty acid metabolism, said method comprising administering an effective amount of a combination of at least one histidine or derivative thereof, at least one glycine or derivative thereof, and lysine.

In some embodiments, the method is for treating or preventing at least one physical state selected from the group consisting of high HOMA-IR, high fasting glucose and high insulin, said method comprising administering an effective amount of a combination of at least one histidine or derivative thereof, at least one glycine or derivative thereof, and lysine.

In some embodiments, the method is for treating or preventing at least one physical state selected from the group consisting of oxidative stress, a condition associated with oxidative stress, or a condition associated with a reduced level of glutathione, said method comprising administering an effective amount of a combination of at least one histidine or derivative thereof, at least one glycine or derivative thereof, and lysine.

In some embodiments, the method is for treating or preventing at least one physical state selected from the group consisting of high body weight gain and associated disturbed glucose metabolism during growth and development, high body fat gain and associated disturbed glucose metabolism during growth and development, high central adiposity and associated disturbed glucose metabolism during growth and development, said method comprising administering an effective amount of a combination of at least one histidine or derivative thereof, at least one glycine or derivative thereof, and lysine.

In another embodiment, the method is for enhancing metabolization of reactive oxygen species, improving glucose control and/or improving muscle function in an individual with at least one of obesity or diabetes, said method comprising administering to the individual an effective amount of a combination of at least one histidine or derivative thereof, at least one glycine or derivative thereof and at least one lysine or derivative thereof.

In another embodiment, the method is for enhancing metabolization of reactive oxygen species, improving glucose control and/or improving muscle function in an individual with at least one of obesity or diabetes, said method comprising administering to the individual an effective amount of a combination of at least one histidine or derivative thereof, at least one glycine or derivative thereof and at least one arginine or derivative thereof.

In another embodiment, the method is for enhancing metabolization of reactive oxygen species, improving glucose control and/or improving muscle function in an individual with at least one of obesity or diabetes, said method comprising administering to the individual an effective amount of a combination of at least one histidine or derivative thereof, at least one glycine or derivative thereof and at least one N-acetyl-cysteine or derivative thereof.

In another embodiment, the method is for improving mitochondrial function in an individual with sarcopenia, said method comprising administering to the individual an effective amount of a combination of at least one histidine or derivative thereof, at least one glycine or derivative thereof and at least one lysine or derivative thereof. The individual with sarcopenia can be otherwise healthy or obese sarcopenic.

In another embodiment, the method is for improving mitochondrial function in an individual with sarcopenia, said method comprising administering to the individual an effective amount of a combination of at least one histidine or derivative thereof, at least one glycine or derivative thereof and at least one arginine or derivative thereof. The individual with sarcopenia can be otherwise healthy or obese sarcopenic.

In another embodiment, the method is for improving mitochondrial function in an individual with sarcopenia, said method comprising administering to the individual an effective amount of a combination of at least one histidine or derivative thereof, at least one glycine or derivative thereof and at least one N-acetyl-cysteine or derivative thereof. The individual with sarcopenia can be otherwise healthy or obese sarcopenic.

In an embodiment, the method is for treating or preventing at least one physical state selected from the group consisting of deleterious effects of type I diabetes, type II diabetes, complications from diabetes, insulin resistance, metabolic syndrome, dyslipidemia, overweight, obesity, raised cholesterol levels, raised triglyceride levels, elevated fatty acid levels, fatty liver disease, cardiovascular disease, myopathy such as statin-induced myopathy, non-alcoholic steatohepatitis, hypertension, atherosclerosis/coronary artery disease, myocardial damage after stress.

In an embodiment, the at least one glycine or derivative thereof is selected from the group consisting of L-glycine, L-glycine ethyl ester, D-Allylglycine; N-[Bis(methylthio)methylene]glycine methyl ester; Boc-allyl-Gly-OH (dicyclohexylammonium) salt; Boc-D-Chg-OH; Boc-Chg-OH; (R)—N-Boc-(2′-chlorophenyl)glycine; Boc-L-cyclopropylglycine; Boc-L-cyclopropylglycine; (R)—N-Boc-4-fluorophenylglycine; Boc-D-propargylglycine; Boc-(S)-3-thienylglycine; Boc-(R)-3-thienylglycine; D-a-Cyclohexylglycine; L-a-Cyclopropylglycine; N-(2-fluorophenyl)-N-(methylsulfonyl)glycine; N-(4-fluorophenyl)-N-(methylsulfonyl)glycine; Fmoc-N-(2,4-dimethoxybenzyl)-Gly-OH; N-(2-Furoyl)glycine; L-a-Neopentylglycine; D-Propargylglycine; sarcosine; Z-a-Phosphonoglycine trimethyl ester, and mixtures thereof.

In an embodiment, the combination of at least one histidine or derivative thereof, at least one glycine or derivative thereof, and at least one additional active agent, such as N-acetyl-cysteine, lysine, or arginine is administered orally.

In an embodiment, the combination of at least one histidine or derivative thereof, at least one glycine or derivative thereof, and at least one additional agent, such as N-acetyl-cysteine, lysine, or arginine is administered in a food product.

In an embodiment, the combination of at least one histidine or derivative thereof, at least one glycine or derivative thereof, and at least one additional agent, such as N-acetyl-cysteine, lysine, or arginine is administered in a composition that comprises a dipeptide that provides at least a portion of the at least one glycine or derivative thereof and the at least one N-acetylcysteine or derivative thereof.

In an embodiment, the at least one histidine or functional derivative thereof, the at least one glycine or derivative thereof, and the at least one lysine or derivative thereof are administered in the same composition.

In an embodiment, the at least one histidine or functional derivative thereof, the at least one glycine or derivative thereof, and the at least one arginine or derivative thereof are administered in the same composition.

In an embodiment, the at least one histidine or derivative thereof, the at least one glycine or derivative thereof, and the at least one N-acetylcysteine or derivative thereof are administered in the same composition.

In an embodiment, the at least one histidine or derivative thereof, the at least one glycine or derivative thereof, and the at least one lysine or derivative thereof are administered in a different composition relative to the remainder of the combination.

In an embodiment, the at least one histidine or derivative thereof, the at least one glycine or derivative thereof, and the at least one arginine or derivative thereof are administered in a different composition relative to the remainder of the combination.

In an embodiment, the at least one histidine or derivative thereof, the at least one glycine or derivative thereof, and the at least one N-acetylcysteine or derivative thereof are administered in a different composition relative to the remainder of the combination.

“Weight management” for an adult (e.g., at least eighteen years from birth) means that the individual has approximately the same body mass index (BMI) after one week of consumption of the composition, preferably after one month of consumption of the composition, more preferably after one year of consumption of the composition, relative to their BMI when consumption of the composition was initiated. “Weight management” for younger individuals means that the BMI is approximately the same percentile relative to an individual of a corresponding age after one week of consumption of the composition, preferably after one month of consumption of the composition, more preferably after one year of consumption of the composition, relative to their BMI percentile when consumption of the composition was initiated.

In a related embodiment, a method of weight management in an individual comprises administering to the individual a composition comprising an effective amount of a combination of at least one histidine or derivative thereof, at least one glycine or derivative thereof, and at least one of the following amino acid, lysine, arginine or N-acetyl-cysteine, or derivative thereof.

For example, the composition comprising a combination of a combination of at least one histidine or derivative thereof, at least one glycine or derivative thereof, and at least one of the following amino acid, lysine, arginine or N-acetyl-cysteine, or derivative thereof can be administered to an individual that is managing their weight or undergoing a weight loss program. The weight loss program may include, for example, a weight loss diet (e.g., one or more of a low-fat diet, for example a diet with less than 20% of the calories from fat, preferably less than 15% from fat; a low-carbohydrate diet, for example a diet with less than 20% of the calories from carbohydrates; a low-calorie diet, for example a diet with less calories per day relative to the individual's previous intake before the diet, or a diet with less calories per day relative to an average person of similar body type; or a very low-calorie diet, for example a diet with 800 kcal (3,300 kJ) per day or less). Additionally or alternatively, the weight loss program may include a weight loss training regimen (e.g. endurance and/or strength training).

The method can comprise identifying the individual as being in need of weight management or weight loss and/or identifying the individual as obese or overweight, e.g., before initial administration of the composition.

In each of the compositions and methods disclosed herein, the composition is preferably a food product, including food additives, food ingredients, functional foods, dietary supplements, medical foods, nutraceuticals, or food supplements.

Histidine is an amino acid having the chemical name 2-amino-3-(3H-imidazol-4-yl)propanoic acid. Histidine exists in two enantiomeric forms, L-histidine and D-histidine, as shown below:

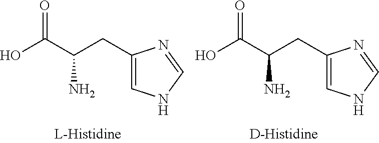

References herein to the generic term “histidine” include any scalemic or racemic mixture of the enantiomers (wherein a scalemic mixture contains the enantiomers in any relative proportions and a racemic mixture contains the enantiomers in the ratio 50:50), as well as L-histidine and/or D-histidine. References herein to individual enantiomers are specific to that enantiomer only. When a scalemic mixture is provided, the mixture preferably comprises more L-histidine than D-histidine, more preferably the mixture comprises mostly L-histidine. For example, the scalemic mixture may comprise at least 60%, more preferably at least 75%, even more preferably at least 90% by weight of L-histidine. References herein to the generic term “histidine” also include all tautomeric forms. Preferably, the histidine is L-histidine and/or a derivative thereof. L-histidine occurs naturally and is readily obtainable from natural sources.

The histidine may be synthesised from suitable starting materials using standard procedures of organic chemistry or may be isolated from natural sources using well known procedures. The synthesis or isolation of particular enantiomers of histidine may be carried out by standard techniques of organic chemistry well known in the art, for example by synthesis from optically active starting materials or by resolution of a racemic form for example by suitable well known techniques.

L-histidine may for example be isolated from any suitable source, such as from meat, poultry, dairy, fish, rice, wheat and rye.

Histidine may be provided as a solid or semi-solid, preferably as a powder.

Any suitable derivative of histidine may be used in the present invention, provided that the derivative is suitable for including in a pharmaceutical composition and provides the desired pharmacological effect as discussed herein. Combinations of histidine and suitable derivatives thereof may be used. The derivatives of histidine may be synthesised from suitable starting materials using standard procedures of organic chemistry or may be isolated from natural sources using well-known procedures.

Suitable derivatives of histidine may be comprised predominantly of a histidine core with minor modifications to functional groups of the histidine core. For example, references herein to derivatives of histidine include compounds derived from histidine (i.e. having a histidine core) in which the carboxylic acid or amino group of the histidine core is derivatised to include a substituent or alternative functional group. For example, suitable derivatives in which the hydroxy group of the carboxylic acid group is derivatised may include an ester (such as an ester formed by the reaction of the carboxylic acid and an alcohol such as methanol, ethanol, isopropanol or butanol). Suitable derivatives in which the amino group is derivatised may include a dialkyl- or trialkyl-amine (such as a dialkyl- or trialkyl-amine formed by the reaction of the amino group and an alkyl-halide).

Further suitable derivatives of histidine include peptides of histidine, such as peptides including 2 or more histidine units, for example from 2 to 20 histidine units, particularly from 2 to 10 histidine units or from 7 to 10 histidine units, or for example 20 or more histidine units. Particular such derivatives may be di- and tri-peptides of histidine.

Further suitable derivatives of histidine include peptides of histidine and one or more additional amino acids, such as peptides including 2 or more histidine/additional amino acid units, for example from 2 to 20 histidine/additional amino acid units, particularly from 2 to 10 histidine/additional amino acid units or from 7 to 10 histidine/additional amino acid units, or for example 20 or more histidine/additional amino acid units. Particular such derivatives may be di- and tri-peptides, such as a dipeptide of histidine and beta-alanine (otherwise known as carnosine).

Further suitable derivatives may include, for example, pharmaceutically-acceptable salts or pro-drugs of histidine and the functionalised compounds or polypeptides as discussed above.

By the term pro-drug we mean a compound that is broken down in a subject, for example in a warm-blooded animal such as man, to release the histidine and/or the derivative thereof.

Examples of pro-drugs may include in vivo cleavable ester derivatives such as those described above. Suitable pharmaceutically-acceptable salts and pro-drugs are based on reasonable medical judgement as being suitable for administration to a subject, for example a warm-blooded animal such as man, without undesirable pharmacological activities and without undue toxicity. Examples of suitable pharmaceutically-acceptable salts include acid-addition salts with an inorganic or organic acid such as hydrochloric, hydrobromic, sulfuric, trifluoroacetic, citric or maleic acid.

In all aspects of the present invention as described herein any derivative of histidine is preferably selected from one or more of a peptide of histidine (particularly di- and tri-peptides of histidine), a peptide of histidine and one or more additional amino acids (particularly di- and tri-peptides, for example carnosine), and a pharmaceutically-acceptable salt of histidine.

More preferably, any derivative of histidine is selected from one or more of a di- or tri-peptide of histidine, a di- or tri-peptide of histidine and one or more additional amino acids (for example carnosine), and a pharmaceutically-acceptable salt of histidine. Even more preferably, any derivative of histidine is a pharmaceutically-acceptable salt of histidine.

Most preferably, in all aspects of the present invention as described herein, the active ingredient is histidine, more preferably L-histidine. In other words, the present invention preferably provides histidine (more preferably L-histidine) for use in maintaining and/or improving barrier function of the skin of a subject and for the prevention of a skin disorder (particularly an inflammatory skin disease, more particularly a chronic inflammatory skin disease, and even more particularly atopic dermatitis) as described herein, as well as pharmaceutical compositions and nutritional products comprising histidine (more preferably L-histidine) as described herein.

Lysine, its isomeric forms (L- or D- either alone or, in various combinations amongst themselves), salts thereof and the short oligomers (most preferably up to M.W. 1000) and salts thereof, derivatives (e.g. acetyl-lysine/oligo-lysine) as the active ingredient(s) (with or without one or more additive(s)) may be used according to the invention.

The glycine is preferably L-glycine and/or L-glycine ethyl ester. Non-limiting examples of suitable glycine functional derivatives include D-Allylglycine; N-[Bis(methylthio)methylene]glycine methyl ester; Boc-allyl-Gly-OH (dicyclohexylammonium) salt; Boc-D-Chg-OH; Boc-Chg-OH; (R)—N-Boc-(2′-chlorophenyl)glycine; Boc-L-cyclopropylglycine; Boc-L-cyclopropylglycine; (R)—N-Boc-4-fluorophenylglycine; Boc-D-propargylglycine; Boc-(S)-3-thienylglycine; Boc-(R)-3-thienylglycine; D-a-Cyclohexylglycine; L-a-Cyclopropylglycine; N-(2-fluorophenyl)-N-(methylsulfonyl)glycine; N-(4-fluorophenyl)-N-(methylsulfonyl)glycine; Fmoc-N-(2,4-dimethoxybenzyl)-Gly-OH; N-(2-Furoyl)glycine; L-a-Neopentylglycine; D-Propargylglycine; sarcosine; Z-a-Phosphonoglycine trimethyl ester, and a mixture thereof.

In an embodiment, both the glycine and the N-acetylcysteine may be provided in a dipeptide, such as N-acetylcysteinylglycine or cysteinylglycine.

The composition can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

The N-acetylcysteine or functional derivative thereof can be administered in an amount of about 0.1-100 milligram (mg) of N-acetylcysteine or functional derivative thereof per kilogram (kg) of body weight of the subject. The glycine or functional derivative thereof can be administered in an amount of about 0.1-100 milligram (mg) of glycine or functional derivative thereof per kilogram (kg) of body weight of the subject.

The compositions disclosed herein may be administered to the subject orally or parenterally. Non-limiting examples of parenteral administration include intravenously, intramuscularly, intraperitoneally, subcutaneously, intraarticularly, intrasynovially, intraocularly, intrathecally, topically, and inhalation. As such, non-limiting examples of the form of the composition include natural foods, processed foods, natural juices, concentrates and extracts, injectable solutions, microcapsules, nano-capsules, liposomes, plasters, inhalation forms, nose sprays, nosedrops, eyedrops, sublingual tablets, and sustained-release preparations.

The compositions disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms, such as tablets, capsules, powders, granules, ointments, solutions, suppositories, injections, inhalants, gels, microspheres, and aerosols.

As such, administration of the composition can be achieved in various ways, including oral, buccal, rectal, parenteral, intraperitoneal, intradermal, transdermal, and intratracheal administration. The active agent may be systemic after administration or may be localized by the use of regional administration, intramural administration, or use of an implant that acts to retain the active dose at the site of implantation.

In pharmaceutical dosage forms, the compounds may be administered as their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in an aerosol formulation to be administered by inhalation. For example, the compounds can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds can be administered rectally by a suppository. The suppository can include a vehicle such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition. Similarly, unit dosage forms for injection or intravenous administration may comprise the compounds in a composition as a solution in sterile water, normal saline or another pharmaceutically acceptable carrier, wherein each dosage unit, for example, mL or L, contains a predetermined amount of the composition containing one or more of the compounds.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

In one aspect of the invention, the change in diet is associated with physical activity program management. The physical activity program should be adapted to body composition, medical conditions and age of the subjects, aiming at weight loss or weight management, and improvement of body fat mass and lean mass for optimal glucose management outcome.

For instance, the solution may be part of a Physical Activity Program which use all opportunities for students to be physically active, meet the nationally-recommended minutes of physical activity each day (e.g. 60 minutes of moderate to vigorous physically activity each day). For instance, the program may follow public health guidelines for physical activity for children and young people (as an example, National institute for health and care excellence, UK: https://www.nice.org.uk/guidance).

### EXAMPLES

### Example 1

Methods Used During the Study

Study Population

The EarlyBird Diabetes Study incorporates a 1995/1996 birth cohort recruited in 2000/2001 when the children were 5 years old (307 children, 170 boys). The collection of data from the Early Bird cohort is composed of several clinical and anthropometric variables measured on an annual basis from the age of 5 to the age of 16. The study was conducted in accordance with the ethics guidelines of the Declaration of Helsinki II; ethics approval was granted by the Plymouth Local Research Ethics Committee (1999), and parents gave written consent and children verbal assent.

Following a good cohort retention at an age when some young people will begin moving from home to start their own lives. A follow-up study was prepared from June 2013 and began study visits in February 2015 until summer 2016. A total of 178 Earlybird participants completed this follow-up visit as an adult (average age of 20 years old) where data were collected using an adapted study protocol.

Anthropometric Parameters

BMI was derived from direct measurement of height (Leicester Height Measure; Child Growth Foundation, London, U.K.) and weight (Tanita Solar 1632 electronic scales), performed in blind duplicate and averaged. BMI SD scores were calculated from the British 1990 standards.

Physical activity was measured annually from 5 years by accelerometry (Acti-Graph [formerly MTI/CSA]). Children were asked to wear the accelerometers for 7 consecutive days at each annual time point, and only recordings that captured at least 4 days were used.

Resting energy expenditure was measured by indirect calorimetry using a ventilated flow through hood technique (Gas Exchange Measurement, Nutren Technology Ltd, Manchester, UK). Performance tests reportedly show a mean error of 0.3±2.0% in the measurement of oxygen consumption and 1.8±1% in that of carbon dioxide production. Measurements were performed in a quiet thermoneutral room (20° C.) after overnight fasting period of at least 6 hours, to minimize any effect attributable to the thermic effect of food. Data were collected for a minimum of 10 minutes and the respiratory quotient (RQ) was calculated as an indicator of basal metabolic rate (BMR).

Clinical Parameters

Peripheral blood was collected annually into EDTA tubes after an overnight fast and stored at −80° C. Insulin resistance (IR) was determined each year from fasting glucose (Cobas Integra 700 analyzer; Roche Diagnostics) and insulin (DPC IMMULITE) (cross-reactivity with proinsulin, 1%) using the homeostasis model assessment program (HOMA-IR), which has been validated in children.

Serum Metabonomics

400 μL of blood serum was mixed with 200 μL of deuterated phosphate buffer solution 0.6 M KH2PO4, containing 1 mM of sodium 3-(trimethylsilyl)-[2,2,3,3-2H4]-1-propionate (TSP, chemical shift reference δH=0.0 ppm). 550 μL of the mixture was transferred into 5 mm NMR tubes.

1H NMR metabolic profiles of serum samples were acquired with a BrukerAvance III 600 MHz spectrometer equipped with a 5 mm cryoprobe at 310 K (Bruker Biospin, Rheinstetten, Germany) and processed using TOPSPIN (version 2.1, Bruker Biospin, Rheinstetten, Germany) software package as reported previously. Standard 1H NMR one-dimensional pulse sequence with water suppression, Carr-Purcell-Meiboom-Gill (CPMG) spin-echo sequence with water suppression, and diffusion-edited sequence were acquired using 32 scans with 98K data-points. The spectral data (from δ 0.2 to δ 10) were imported into Matlab software with a resolution of 22K data-points (version R2013b, the Mathworks Inc, Natwick Mass.) and normalized to total area after solvent peak removal. Poor quality or highly diluted spectra were discarded from the subsequent analysis.

1H-NMR spectrum of human blood plasma enables the monitoring of signals related to lipoprotein bound fatty acyl groups found in triglycerides, phospholipids and cholesteryl esters, together with peaks from the glyceryl moiety of triglycerides and the choline head group of phosphatidylcholine. This data also covers quantitative profiling of major low molecular weight molecules present in blood. Based on internal database, representative signals of metabolites assignable on 1H CPMG NMR spectra were integrated, including asparagine, leucine, isoleucine, valine, 2-ketobutyric acid, 3-methyl-2-oxovaleric acid, alpha-ketoisovaleric acid, (R)-3-hydroxybutyric acid, lactic acid, alanine, arginine, lysine, acetic acid, N-acetyl glycoproteins, 0-acetyl glycoproteins, acetoacetic acid, glutamic acid, glutamine, citric acid, dimethylglycine, creatine, citrulline, trimethylamine, trimethylamine N-oxide, taurine, proline, methanol, glycine, serine, creatinine, histidine, tyrosine, formic acid, phenylalanine, threonine, and glucose. In addition, in diffusion edited spectra, signals associated to different lipid classes were integrated, including phospholipids containing choline, VLDL subclasses, unsaturated and polyunsaturated fatty acid. The signals are expressed in arbitrary units corresponding to a peak area normalized to total metabolic profiles, which is representative of relative change in metabolite concentration in the serum.

Mass-Spectrometry Based Determination of Serum Amino Acids

Blood serum amino acids were quantified on selected samples using an in-house automated quantification method of amino acids in human plasma and serum by UPLC-MS/MS. Briefly, following a step of precipitation, derivatization and dilution, samples are submitted to liquid chromatography (Acquity I-class, Waters) coupled to mass spectrometry analysis (Xevo TQ-XS triple quadrupole, Waters). For chromatographic separation, a gradient composed a mobile phase of Ammonium Formate (Ammonium formate 0.55 g/L in water at 0.1% formic acid), and a second mobile phase of acetonitrotion (acetonitrile 0.1% formic acid). Analyte concentrations are calculated from peaks area ratio of the compounds to their corresponding internal standards. Results are expressed in μM. Peaks are integrated using AA_quantitationmeth in TargetLynx functionality included in MassLynx software.

Statistics

The distribution of the outcome variable, IR, was skewed and so log-transformed for analysis. For both pilot and main study analyses, using data at all ages simultaneously, mixed effects modelling was used to assess the association between IR (HOMA-IR) and individual metabolites, taking into account age, BMI sds, physical activity and pubertal timing (APHV). Random intercepts were included as well as age (categorized to allow for non-linear change in IR over time), gender, DEXA % fat, APHV, MVPA (number of minutes spent in moderate-vigorous physical activity) and individual metabolites (in separate models) as fixed effects.

The present inventors carried out a first study on a sub-set of 40 of the participants from 5y to 14y (Pilot study), and assessed repeatability on another subset of 150 participants from 5y to 16y (Main study). In the Pilot study, 40 subjects were chosen on the basis of having a complete set of samples available for analysis at each time-point between 5y and 14y (20 boys), having been stratified by IR at 5 and 14 years. In the Main study, 150 subjects were chosen to include all of those who had shown impaired fasting glucose at one or more time-points during the course of the study. Only 28 children were common in the two studies. The subjects who had shown impaired fasting glucose were matched for gender resulting in the selection of 105 boys and 45 girls.

To assess further which IR-associated metabolite may be an early indicator of IR trajectories, the present inventors stratified the main study population according to low or high IR status over the 14-16 year age range. Arbitrarily the 91st centile for the HOMA-IR distribution was employed as a threshold to define children with high IR status. Here, mixed effects modelling was used to assess the association between IR and individual metabolites. Modelling was carried out in R software (www.R-project.org) using the Imer function in the package Ime4 (Bates, Maechler et al. 2015) and p-values calculated using the Satterthwaite approximation implemented in the ImerTest package (Kuznetsova, Brockhoff et al. 2016).

### Example 2

Measurement of Metabolite Concentrations

Clinical and anthropometrics characteristics of the children in the pilot study at 5 and 14 years are summarized in Table 1 and those in the main study at 5y, 14y and 16y in Table 2. In both genders there was a decrease in HOMA-IR up to around 8y, which was followed by an increase through puberty, this trend being dependent on timing of peak height velocity (age*APHV interaction p<0.001). IR was also positively associated with BMI sds (p<0.001).

TABLE 1
Characteristics of the cohort at 5 y and 14 y by gender.

| | | | Boys | | Girls | |
|---|---|---|---|---|---|---|
| Age (years) | 5 | y | 5.1 | (4.8-5.3) | 4.8 | (4.7-5.0) |
| | 14 | y | 13.9 | (13.6-14.1) | 13.8 | (13.7-14.1) |
| BMI sds | 5 | y | −0.04 | (−0.50-0.72) | 0.40 | (0.04-0.80) |
| | 14 | y | 0.43 | (−0.13-1.29) | 0.78 | (−0.05-1.48) |
| Moderate-vigorous | 5 | y | 46.1 | (34.1-67.1) | 55.1 | (44.9-62.5) |
| physical activity | 14 | y | 52.4 | (29.7-77.0) | 42.7 | (29.3-51.4) |
| (minutes/day) | | | | | | |
| Age at peak height | | | 13.4 | (12.9-13.8) | 11.9 | (11.1-12.5) |
| velocity (years) | | | | | | |
| IR (HOMA2-IR) | 5 | y | 0.47 | (0.37-0.84) | 0.85 | (0.34-1.02) |
| | 14 | y | 1.25 | (0.56-1.63) | 0.98 | (0.78-2.23) |

Data are median (interquartile range)

TABLE 2
Characteristics of the main cohort at 5 y, 14 y and 16 y by gender.

| | | | Boys | | Girls | |
|---|---|---|---|---|---|---|
| Age (years) | 5 | y | 4.8 | (4.7-5.0) | 4.9 | (4.8-5.1) |
| | 14 | y | 13.8 | (13.6-14.0) | 13.9 | (13.8-14.0) |
| | 16 | y | 15.8 | (15.6-16.0) | 15.9 | (15.8-16.1) |
| BMI sds | 5 | y | 0.09 | (−0.48-0.80) | 0.36 | (−0.49-1.16) |
| | 14 | y | 0.32 | (−0.64-0.98) | 0.85 | (0.21-1.60) |
| | 16 | y | 0.44 | (−0.31-1.32) | 0.70 | (0.03-1.60) |
| Moderate-vigorous | 5 | y | 53.1 | (41.6-64.1) | 40.1 | (31.9-51.6) |
| physical activity | 14 | y | 44.4 | (30.7-63.14) | 35.3 | (18.4-50.4) |
| (minutes/day) | 16 | y | 43.9 | (22.5-58.6) | 27.0 | (16.8-40.9) |
| Age at peak height | | | 13.0 | (12.8-13.4) | 11.6 | (10.8-12.3) |
| velocity (years) | | | | | | |
| IR (HOMA2-IR) | 5 | y | 0.49 | (0.22-0.74) | 0.76 | (0.60-1.00) |
| | 14 | y | 1.00 | (0.79-1.47) | 1.47 | (1.00-1.70) |
| | 16 | y | 0.70 | (0.23-1.16) | 0.84 | (0.23-1.18) |

Data are median (interquartile range)

Using data at all ages simultaneously, mixed effects modelling was applied to assess the association between HOMA-IR and individual metabolites. In the pilot study several metabolites including BCAAs, lipids and other amino acids showed a significant association (p<0.05) with HOMA-IR in longitudinal models, independently of BMI sds, physical activity and pubertal timing as shown in Table 3. The Table 4 reports the outcomes of the same analysis conducted on the main study cohort.

TABLE 3
Estimates and p-values from mixed effects models
examining the association between metabolites and
insulin resistance in the pilot study (n = 40)

| | Metabolic | | | |
|---|---|---|---|---|
| Metabolite | pathway | Coef | SE | p-value |
| Citrate | Glycolysis | −0.132 | 0.028 | 0.000003 |
| | related | | | |
| Phospholipids | Lipid | −0.131 | 0.031 | 0.000038 |
| 3-D-hydroxybutyrate | Ketone bodies | −0.106 | 0.027 | 0.000126 |
| Creatine | Organic acid | −0.095 | 0.029 | 0.001101 |
| Lipid (mainly LDL, | Lipid | 0.087 | 0.028 | 0.002272 |
| fatty acid (CH₂)ₙ | | | | |
| moieties) | | | | |
| Lactate | Glycolysis | 0.067 | 0.025 | 0.007194 |
| | related | | | |
| Lysine | Amino acids | −0.060 | 0.027 | 0.025594 |
| Histidine | Amino acids | −0.057 | 0.027 | 0.036010 |
| Lipid (mainly VLDL, | Lipid | 0.043 | 0.023 | 0.061526 |
| fatty acid (CH₂) | | | | |
| moieties) | | | | |
| Dimethylglycine | Amino acids | −0.046 | 0.026 | 0.082072 |
| Glycine | Amino acids | 0.007 | 0.030 | 0.818118 |

TABLE 4
Estimates and p-values from mixed effects models
examining the association between metabolites and
insulin resistance in the main study (n = 150)

| | Metabolic | | | p-value |
|---|---|---|---|---|
| Metabolites | pathway | Coef | SE | (unadjusted) |
| Citrate | Glycolysis | −0.188 | 0.021 | <0.0001 |
| | related | | | |
| Phospholipids | Lipid | −0.066 | 0.024 | 0.0067 |
| 3-D-hydroxybutyrate | Ketone bodies | −0.092 | 0.018 | <0.0001 |
| Creatine | Organic acid | −0.142 | 0.023 | <0.0001 |
| Lipid (mainly LDL, | Lipid | 0.133 | 0.023 | <0.0001 |
| fatty acid (CH2)n | | | | |
| moieties) | | | | |
| Lactate | Glycolysis | 0.101 | 0.019 | <0.0001 |
| | related | | | |
| Lysine | Amino acids | −0.139 | 0.02 | <0.0001 |
| Histidine | Amino acids | −0.124 | 0.022 | <0.0001 |
| Lipid (mainly VLDL, | Lipid | −0.12 | 0.022 | <0.0001 |
| fatty acid (CH2) | | | | |
| moieties) | | | | |
| Dimethylglycine | Amino acids | −0.118 | 0.021 | <0.0001 |
| Glycine | Amino acids | −0.08 | 0.026 | 0.0023 |

The analysis has highlighted the importance of specific metabolites in amino acid, ketone body, glycolysis and fatty acid metabolism, in describing the variations of HOMA-IR throughout the childhood. This is believed to be the first report of a metabolic contribution of specific metabolic processes to overall insulin metabolism variations in a longitudinal and continuous manner.

Central Energy-Related Metabolites

In the pilot and main study cohorts, mixed effects modelling described inverse associations of IR with citrate and 3-D-hydroxybutyrate overall (p<0.001), and positive associations of IR with lactate (p<0.01). The analysis of the main study describes statistically significant year-on-year correlations for citrate ranging from r=0.28 to r=0.66 p<0.05), while those for 3-D-hydroxybutyrate were not significant before 8y and then ranged from r=0.21 to r=0.58 (p<0.05). In the main study, citrate was inversely correlated with IR at each cross-sectional time-point between 5y and 16y (correlations ranging from r=−0.21 to r=−0.52, p<0.05). 3-D-hydroxybutyrate showed inverse cross-sectional correlations (correlations ranging from r=−0.21 to r=−0.53, p<0.05) up to age. Lactate showed positive cross-sectional correlations with IR (correlations ranging from r=0.13 to r=0.45, p<0.05).

Amino Acid Metabolism

Mixed effects modelling identified statistically significant inverse associations between histidine, creatine and lysine with IR (p<0.05), which replicated in the main study (p<0.001). Each metabolite also showed inverse cross-sectional correlations with IR, particularly so between 9y and 14y (correlations ranging from r=−0.17 to r=−0.46, p<0.05).

Lipid Related Metabolites Associated with IR

¹H-NMR spectrum of human blood serum enables the monitoring of signals related to lipoprotein bound fatty acyl groups found in triglycerides, phospholipids and cholesteryl esters, together with peaks from the glyceryl moiety of triglycerides and the choline head group of phosphatidylcholine.

Here, signals derived from the methyl fatty acyl groups in phospholipids containing choline showed inverse associations with IR, whereas signals derived from the methyl fatty acyl groups in LDL particles showed positive associations with IR. The lipid signals were highly correlated with each other (r>0.8 between 5y and 13y, and r=0.6 at 14y). These associations were also found in the main study both in the mixed effects model and at individual time-points. Cross-sectional associations between IR and phospholipids were inverse and statistically significant from age 7y (correlations ranged from r=−0.19 to r=−0.54), whereas those between IR and fatty acyl groups in LDL particles were positive and statistically significant between 7y and 14y (correlations ranging from r=0.24 to r=0.41). Whilst not significant in the pilot study (p=0.06), fatty acyl groups in VLDL particles showed a positive association with IR in the mixed effects model, consistent with cross-sectional association which were positive and statistically significant at age 5y and between 7y and 14y (correlations ranging from r=0.25 to r=0.46).

### Example 3

Metabolites Indicative of Higher HOMA-IR at Adolescence

For each metabolite showing a significant association overtime with IR, the present inventors assessed further if their serum concentration was informative of low or high IR status over the 14-16 year age range. Arbitrarily the 91^{st} centile for the HOMA-IR distribution was employed as a threshold to define children with high IR status (Table 5). It was further explored—amongst the metabolites contributing the most to HOMA-IR variations in childhood—which ones may be an earlier and a more indicative indicator of higher HOMA-IR at adolescence.

TABLE 5
Estimates and p-values from mixed effects models examining
the association between metabolites and HOMA-IR groups

| | p-value | p-value |
|---|---|---|
| | Group | Age:group |
| Metabolites | difference | interaction |
| Citrate | <0.01 | NS |
| Phospholipids | <0.001 | NS |
| 3-D-hydroxybutyrate | <0.05 | NS |
| Lipid (mainly LDL, | <0.01 | NS |
| fatty acid (CH2)n | | |
| moieties) | | |
| Lactate | <0.001 | NS |
| Histidine | <0.05 | NS |
| Lysine | <0.01 | NS |
| Lipid (mainly VLDL, | <0.01 | NS |
| fatty acid (CH2) | | |
| moieties) | | |
| Creatine | 0.56 | <0.05 |
| Glycine | <0.01 | NS |
| Creatine:Glycine ratio | <0.05 | <0.001 |

Therefore, amongst the most influential biochemical species contributing to high HOMA-IR in childhood, the analysis indicates that:
Mixed effects modelling identified a significant positive association overtime between high IR status and lactate, fatty acyl groups in LDL and VLDL particles, and creatine:glycine ratio.
A significant negative association was found overtime between high IR status and citrate, histidine, 3-D-hydroxybutyrate, glycine, creatine, lysine and phospholipids.
Fat mass (waist circumference) was also a statistically significant variable increased in high IR group over time (p<0.001), with a significant interaction between age and group (p<0.001).

Significant increases in the annual incidence of both type 1 diabetes and type 2 diabetes among youths (aged 10 to 19 years old) in the United States have been recently reported by Mayer-Davis et al. (Incidence Trends of Type 1 and Type 2 Diabetes among Youths, 2002-2012, The New England Journal of Medicine, 376:1419-1429, 2017). It is well established that variations exist across racial and ethnic groups. As illustrated by Mayer-Davis et al, this includes high relative increases in the incidence of type 2 diabetes in racial and ethnic groups other than non-Hispanic whites in the USA as an example. Variation across demographic subgroups may reflect varying combinations of genetic, environmental, and behavioural factors that contribute to diabetes. Therefore, reference values should be generated accordingly for the proposed markers.

As an example in the present study cohort, fold of changes between groups are determined from the population, and provided at representative ages (Table 6).

TABLE 6
Fold of change (percentage) in high HOMA-IR
subjects compared to reference group

| Age | 8.00 | 9.00 | 10.00 | 11.00 | 12.00 | 13.00 | 14.00 | 15.00 | 16.00 |
|---|---|---|---|---|---|---|---|---|---|
| Lactate | 11.68 | 4.89 | 11.50 | 8.52 | 13.14 | 13.50 | 12.01 | 18.69 | 13.19 |
| Lysine | 1.32 | 0.52 | 6.13 | −2.77 | −4.62 | −0.02 | −6.82 | −3.61 | 2.47 |
| Citric acid | −4.66 | −7.23 | −5.50 | −7.28 | −6.13 | −6.66 | −16.67 | −2.42 | −1.07 |
| Creatine | −10.87 | 0.28 | −3.78 | −13.86 | −6.72 | −0.57 | −10.57 | −3.43 | 2.83 |
| Glycine | −3.83 | −5.92 | −7.94 | −4.48 | −10.02 | −11.62 | −15.75 | −10.49 | −6.74 |
| Histidine | 2.99 | −6.44 | −1.70 | −3.22 | −3.96 | −9.30 | −8.46 | −3.54 | −5.27 |

The population of children overweight or obese at age 5, remains and further developed excessive fat mass gain and body weight gain throughout puberty and adolescence, and have higher HOMA-IR than in other children. In particular, subjects in the 91^{st} centile of HOMA-IR at adolescence have a particularly marked lower histidine concentration in serum from the age of 9, which corresponded to the period where IR trajectories diverged between groups. They also show a higher body fat and central adiposity (waist circumference) throughout childhood. The status in histidine is negatively associated with C-reactive protein levels at each age for the Earlybird population.

Our results also describe a remodelling of circulating phospholipid species throughout childhood, growth and development. This is a phenomenon well documented in the field of IR and T2D (Szymanska, Bouwman et al. 2012), but not in childhood in relation growth, development and excess of fat gain. The remodelling of phospholipid species is often linked to decreased concentrations in ether-lipids (plasmalogens) in relation to oxidative stress, which have been reported in several diseases; e.g. diabetes mellitus, vascular diseases and obesity.

Histidine and lysine are two representative targets of oxidative modifications. Histidine is extremely sensitive to a metal-catalyzed oxidation, generating 2-oxo-histidine and its ring-ruptured products, whereas the oxidation of lysine generates carbonyl products, such as aminoadipic semialdehyde. On the other hand, both histidine and lysine are nucleophilic amino acids and therefore vulnerable to modification by lipid peroxidation derived electrophiles, such as 2-alkenals, 4-hydroxy-2-alkenals, and ketoaldehydes, derived from lipid peroxidation. Histidine shows specific reactivity toward 2-alkenals and 4-hydroxy-2-alkenals, whereas lysine is a ubiquitous target of aldehydes, generating various types of adducts. Covalent binding of reactive aldehydes to histidine and lysine is associated with the appearance of carbonyl reactivity and antigenicity of proteins. None of these amino acids are reported markers of IR in adult obese subjects.

Since inefficient lipolysis (high basal/low stimulated) was linked to future weight gain and impaired glucose metabolism and may constitute a treatment target (Arner, Andersson et al. 2018), our observation indicate distinctive nutritional requirements during growth and development to promote healthy fat metabolism. In the Earlybird cohort, overweight children at age 5 remain overweight throughout childhood, and will acquire a high IR status from age 10 during pubertal development and development of additional fat mass. Therefore, our observations of negative association with histidine, lysine, and arginine status may be indicative of potential deregulation of oxidative stress and adipocyte lipolysis during growth and development, which are concomitant or contributing to IR development.

From the recommended dietary allowance: 10th Education (National Research Council. 1989. Recommended Dietary Allowances: 10th Edition. Washington, D.C.: The National Academies Press. https://doi.org/10.17226/1349.)
Histidine is an essential amino acid for infants, but was not demonstrated to be required by adults until recently (Cho et al., 1984; Kopple and Swendseid, 1981).
   The requirement for histidine has not been quantified beyond infancy. Requirement values are difficult to establish because deficiency symptoms occur only after long periods of low intake. Kopple and Swendseid (1981) demonstrated that nitrogen balance diminished when histidine intake was less than 2 mg/kg per day, and increased when intake was increased to 4 mg/kg per day. WHO (1985) estimated the probable adult histidine requirement to be between 8 and 12 mg/kg per day by extrapolation from the infant requirement; this estimate is likely to be high, but safe.
   For an adult, requirements in mg/kg per day are between 8-12.
   From (Pencharz and Ball 2006),
      For a child, age 2-10, requirements in mg/kg per day are around 12.
      For a child or adolescent, age 10-14 requirements in mg/kg per day are around 12.
      For an adolescent, age 14-18 requirements in mg/kg per day are around 11.
      For an adult, requirements in mg/kg per day are around 10.
Arginine is synthesized by mammals but not in amounts sufficient to meet the needs of the young of most species. Although it is not believed to be required by the human infant for normal growth, the need for arginine by the premature infant is unknown. When arginine is present in small amounts relative to other amino acids (such as in intravenous solutions or amino acid mixtures), or when liver function is compromised, arginine synthesis may be insufficient for adequate function of the urea cycle (Heird et al., 1972).
From (Pencharz and Ball 2006), requirements for alpha-amino acids are made up of components, namely how much is needed for net incorporation into protein, plus that which is needed for other biological processes. In particular, the following require significant amounts of the amino acids, namely: (a) cysteine, glutamate and glycine for glutathione synthesis; (b) arginine for urea cycle activity, and (c) arginine, glycine and methionine for creatine synthesis.
Lysine is an essential amino acid at every stage of life in humans:
   For an adult, requirements in mg/kg per day are around 12.
   For a child, age 10-12, requirements in mg/kg per day are around 44.
   For a child, age 2, requirements in mg/kg per day are around 64.
   From (Pencharz and Ball 2006),
      For a child, age 2-10, requirements in mg/kg per day are around 35.
      For a child or adolescent, age 10-14 requirements in mg/kg per day are around 35.
      For an adolescent, age 14-18 requirements in mg/kg per day are around 33.
      For an adult, requirements in mg/kg per day are around 30.

### Example 4

Amino Acid and HOMA-IR Status at Adolescence Links to HOMA-IR Status in Adulthood

For both insulin and HOMA-IR, using spearman correlation analysis, the present inventors described how insulin and HOMA-II status in children and adolescents consistently associated statistically significantly with adult status from 11 years throughout childhood and adolescence (Table 7). Therefore, the metabolites contributing the most to HOMA-IR variations in childhood are more indicator of higher HOMA-IR in childhood are relevant markers for high HOMA-IR status in adulthood.

TABLE 7
Spearman Correlation coefficient between subject parameters in
childhood with parameters of the same subjects when aged 20

| Readout | | | | | | |
|---|---|---|---|---|---|---|
| (N = 141) | Age 16 | Age 15 | Age 14 | Age 13 | Age 12 | Age 11 |
| Insulin | 0.2208 | 0.1893 | 0.3109 | 0.2579 | 0.1894 | 0.2126 |
| | (p < 0.001) | (p < 0.05) | (p < 0.001) | (p < 0.001) | (p < 0.05) | (p < 0.01) |
| HOMA1 | 0.2366 | 0.1992 | 0.3142 | 0.2606 | 0.1904 | 0.2235 |
| IR | (p < 0.01) | (p < 0.05) | (p < 0.001) | (p < 0.001) | (p < 0.05) | (p < 0.01) |

Legend: Spearman correlation analysis, data as r coefficient, p value, * 95% CI, ** 99% CI, *** 99.9% CI

In addition, quantitative measures of amino acids were performed in serum samples from the same healthy subjects collected at year 15 and year 20, to provide guidance on healthy reference ranges.

TABLE 8
reference amino acid concentrations
in reference subject groups (N = 168)

Metabolite concentrations in micro molar
(Mean ± SD)

| Marker | Year 15 | Year 20 |
|---|---|---|
| Histidine | 92 ± 14 | 90 ± 15 |
| Lysine | 174 ± 39 | 177 ± 48 |
| Glycine | 304 ± 67 | 279 ± 64 |

### Example 5

Amino Acid Status at Adolescence Correlates with Inflammatory Status

Using spearman correlation analysis, the present inventors described how Lysine, histidine, glycine and creatine:glycine ratio in adolescents associated with a marker of inflammation, C-reactive protein (CRP, Tables 9 and 10). Therefore, the metabolite concentration in childhood which related to HOMA-IR status, are also associated with inflammation status. In particular, Lysine, glycine and histidine, that are negatively associated with HOMA-IR are also negatively associated with CRP, whilst Creatine:glycine ratio is positively correlated with HOMA-IR and inflammatory status.

TABLE 9
Correlation coefficient between metabolites
and C-reactive protein in males

| Age | 13.00 | | 14.00 | |
|---|---|---|---|---|
| Lysine | −0.25 | (p = 0.08) | −0.24 | (p = 0.08) |
| Glycine | −0.32 | (p = 0.02) | −0.30 | (p = 0.03) |
| Creatine:glycine ratio | 0.41 | (p = 0.003) | 0.28 | (p = 0.046) |
| Histidine | −0.21 | (p = 0.15) | −0.33 | (p = 0.01) |

TABLE 10
Correlation coefficient between metabolites
and C-reactive protein in females

| Age | 13.00 | 14.00 |
|---|---|---|
| Lysine | NS | NS |
| Glycine | NS | NS |
| Creatine:glycine | 0.51 | 0.39 |
| ratio | (p = 0.01) | (p = 0.21) |
| Histidine | NS | NS |

Arner, P., D. P. Andersson, J. Backdahl, I. Dahlman and M. Ryden (2018). “Weight Gain and Impaired Glucose Metabolism in Women Are Predicted by Inefficient Subcutaneous Fat Cell Lipolysis.” Cell Metab.
Bates, D., M. Maechler, B. Bolker and S. Walker (2015). “Fitting Linear Mixed-Effects Models Using Ime4.” Journal of Statistical Software 67(1): 1-48.
Feng, R. N., Y. C. Niu, X. W. Sun, Q. Li, C. Zhao, C. Wang, F. C. Guo, C. H. Sun and Y. Li (2013). “Histidine supplementation improves insulin resistance through suppressed inflammation in obese women with the metabolic syndrome: a randomised controlled trial.” Diabetologia 56(5): 985-994.
Kuznetsova, A., P. B. Brockhoff and R. H. B. Christensen (2016). “ImerTest: Tests in Linear Mixed Effects Models. R package version 2.0-30. https://CRAN.R-project.org/package=ImerTest.”
Niu, Y. C., R. N. Feng, Y. Hou, K. Li, Z. Kang, J. Wang, C. H. Sun and Y. Li (2012). “Histidine and arginine are associated with inflammation and oxidative stress in obese women.” Br J Nutr 108(1): 57-61.
Pencharz, P. B. and R. O. Ball (2006). “Amino acid requirements of infants and children.” Nestle Nutr Workshop Ser Pediatr Program 58: 109-116; discussion 116-109.
Pinkney, J., A. Streeter, J. Hosking, M. Mohammod, A. Jeffery and T. Wilkin (2014). “Adiposity, chronic inflammation, and the prepubertal decline of sex hormone binding globulin in children: evidence for associations with the timing of puberty (Earlybird 58).” J Clin Endocrinol Metab 99(9): 3224-3232.
Szymanska, E., J. Bouwman, K. Strassburg, J. Vervoort, A. J. Kangas, P. Soininen, M. Ala-Korpela, J. Westerhuis, J. P. van Duynhoven, D. J. Mela, I. A. Macdonald, R. J. Vreeken, A. K. Smilde and D. M. Jacobs (2012). “Gender-dependent associations of metabolite profiles and body fat distribution in a healthy population with central obesity: towards metabolomics diagnostics.” OMICS 16(12): 652-667.

1. (canceled)

2. A method for use in (i) preventing an increase in insulin resistance and/or (ii) preventing or treating diabetes comprising administering to a subject in need of same a composition comprising a combination of at least one histidine, at least one glycine, and at least one additional agent selected from the group consisting of N-acetyl-cysteine, lysine, and arginine.

3. The composition comprising a combination of at least one histidine, at least one glycine, and lysine for use according to claim 2.

4. A method according to claim 2, wherein said use is for promoting healthy fat metabolism.

5. A method according to claim 2, wherein said use is for treating or preventing at least one additional physical state.

6. A method according to claim 5 wherein said at least one additional physical state is selected from the group consisting of subcutaneous fat cell lipolysis, inefficient lipolysis (high basal/low stimulated), or a condition associated with inefficient lipolysis.

7. A method according to claim 5 wherein said at least one additional physical state is selected from the group consisting of high HOMA-IR, high fasting glucose and high insulin.

8. A method according to claim 5 wherein said at least one additional physical state is selected from the group consisting of oxidative stress, a condition associated with oxidative stress, or a condition associated with a reduced level of glutathione.

9. A method according to claim 5 wherein said at least one additional physical state is selected from the group consisting of high body weight gain and associated disturbed glucose metabolism during growth and development, high body fat gain and associated disturbed glucose metabolism during growth and development, high central adiposity and associated disturbed glucose metabolism during growth and development.

10. A method according to claim 2, wherein said use is for enhancing metabolization of reactive oxygen species, improving glucose control and/or improving muscle function in an individual with at least one of obesity or diabetes.

11. A method according to claim 2, wherein said use is for improving mitochondrial function in an individual with sarcopenia.

12-14. (canceled)

15. A kit comprising at least one histidine, at least one glycine, and at least one additional active agent selected from lysine, arginine or N-acetyl-cysteine, for admixing to form a composition for use in (i) preventing an increase in insulin resistance and/or (ii) preventing or treating diabetes comprising administering to a subject in need of same a composition comprising a combination of at least one histidine, at least one glycine, and at least one additional agent selected from the group consisting of N-acetyl-cysteine, lysine, and arginine.

16. A kit according to claim 15, wherein the admixing occurs in separate containers as two or more liquid solutions or dried powders.
